(19) United States
(12) Patent Application Publication
(10) Pub. No.: US 2024/0393227 A1
(43) Pub. Date: Nov. 28, 2024

(54) METHOD, APPARATUS, ELECTRONIC DEVICE AND MEDIUM FOR DETERMINING ROCK MICROSCOPIC PHYSICAL PARAMETERS

(71) Applicant: Dongying Sansheng Electronic Technology Co., Ltd, Shandong Province (CN)

(72) Inventors: QIUSHI ZHANG, Liaoning Province (CN); SIYU WU, Shandong Province (CN); XUN HAN, Shandong Province (CN); RUOSHUI HAN, Shandong Province (CN)

(21) Appl. No.: 18/671,010

(22) Filed: May 22, 2024

(30) Foreign Application Priority Data
May 24, 2023 (CN) 2023106017537
Apr. 18, 2024 (CN) 2024104800300

(51) Int. Cl. G01N 15/08; G01N 33/24

(52) U.S. Cl. CPC G01N 15/0886; G01N 15/0806; G01N 15/082; G01N 33/241

(57) ABSTRACT

A method for determining rock microscopic physical parameters and an apparatus may determine, based on the acquired macroscopic physical parameters and capillary pressure curve experimental data of a rock, a volume and a saturation of a nonwetting phase fluid injected into different pore throat distribution intervals, and calculate microscopic physical parameters of the rock, through which the properties of the rock, such as a pore structure, storage, seepage and the like can be depicted, thereby facilitating evaluation of hydrocarbon occurrences of a reservoir and formulation of a reasonable hydrocarbon field development strategy.

### CROSS-REFERENCE TO RELATED APPLICATION AND CLAIM OF PRIORITY

This application claims the benefit under 35 USC § 119 of Chinese Patent Applications Nos. 2023106017537, filed on May 24, 2023, and 2024104800300 filed on Apr. 18, 2024, in the China Intellectual Property Office, the entire disclosure of which are incorporated herein by reference for all purposes.

### BACKGROUND

### 1. Technical Field

The implementations of the present disclosure relate to the technical field of hydrocarbon field reservoir evaluation and hydrocarbon field development. More specifically, the present disclosure relates to a method, an apparatus, an electronic device and a medium for determining rock microscopic physical parameters.

### 2. Background Art

Hydrocarbon reservoirs rely on rocks to store and transport hydrocarbons, and therefore, macroscopic physical parameters of the rock, such as permeability, porosity and fluid saturation, influence occurrence and production of the hydrocarbons. On the microscopic level, the microscopic pore structure of the rock directly influences reservoir and seepage characteristics and the like of the rock. Even with the same values of permeability, porosity, fluid saturation and the like on the macroscopic level, rocks of different microscopic pore structures will have different reservoir and seepage characteristics and the like, which indicates that the current rock macroscopic physical parameters have certain defects. Therefore, how to determine proper physical parameters for representing properties of microscopic pores in the rock has an important significance for precise evaluation of hydrocarbon occurrences in the reservoir, study of the fluid seepage rule, formulation of a reasonable development strategy, and the like.

In the prior art, the pore structure of the rock is mainly characterized by physical parameters such as a pore radius median, a displacement pressure, a pore throat radius frequency distribution and the like, but these physical parameters focus on local or average characteristics of the microscopic pore structure, making it difficult to establish direct connection with macroscopic physical parameters (such as permeability, porosity, fluid saturation and the like), or to precisely describe reservoir and seepage characteristics of the microscopic pores. At present, there is a lack of a proper method for determining microscopic physical parameters, making it hard to precisely depict the microstructure, reservoir, seepage and other properties of the rock, and thereby seriously restricting precise evaluation and efficient development of the hydrocarbon field.

In view of this, it is desirable to provide a method for determining physical parameters that can not only reflect macroscopic reservoir and seepage characteristics of the rock, but also characterize a pore structure of the rock.

### SUMMARY

To address one or more of the technical problems mentioned above, the present disclosure provides a method, an apparatus, an electronic device and a medium for determining rock microscopic physical parameters.

According to a first aspect of the present disclosure, there is provided a method for determining rock microscopic physical parameters, including the steps of: acquiring macroscopic physical parameters of a rock, where the macroscopic physical parameters includes an apparent volume, a pore volume, a porosity, a permeability and a fluid saturation of the rock; acquiring capillary pressure curve experimental data of the rock, where the capillary pressure curve experimental data includes a capillary pressure, an accumulated volume of a nonwetting phase fluid, and/or a saturation of a nonwetting phase fluid; based on the capillary pressure curve experimental data of the rock, determining an accumulated volume of a nonwetting phase fluid at two end points of different pore radius distribution intervals, and/or determining a saturation of the nonwetting phase fluid at the two end points of the different pore radius distribution intervals; calculating the rock microscopic physical parameters based on the macroscopic physical parameters of the rock, the accumulated volume of a nonwetting phase fluid injected at the two end points of the different pore radius distribution intervals of the rock, and/or a saturation of the nonwetting phase fluid injected at the two end points of the different pore radius distribution intervals of the rock, where the microscopic physical parameters includes a microscopic pore volume, a microscopic porosity, a microscopic tortuosity, a microscopic permeability, a microscopic fluid saturation, and/or a microscopic efficiency of mercury withdrawal of the rock, where microscopic pores include microscopic pores with saturated nonwetting phase fluid and microscopic pores with minimum unsaturated nonwetting phase fluid; and where calculating the rock microscopic physical parameters includes dividing the rock into a plurality of microscopic pores.

According to a second aspect of the present disclosure, there is provided an apparatus for determining rock microscopic physical parameters, including: a first acquisition unit configured to acquire macroscopic physical parameters of a rock, where the macroscopic physical parameters includes an apparent volume, a pore volume, a porosity, a permeability and a fluid saturation of the rock; a second acquisition unit configured to acquire capillary pressure curve experimental data of the rock, where the experimental data includes a capillary pressure, an accumulated volume of a nonwetting phase fluid, and/or a saturation of a nonwetting phase fluid; a third acquisition unit configured to acquire an accumulated volume of a nonwetting phase fluid at two end points of different pore radius distribution intervals of the rock, and/or a saturation of the nonwetting phase fluid at the two end points of the different pore radius distribution intervals of the rock; and a logical computation unit configured to determine the rock microscopic physical parameters based on the macroscopic physical parameters of the rock, the accumulated volume of the nonwetting phase fluid at the two end points of the different pore radius distribution intervals of the rock, and/or the saturation of the nonwetting phase fluid at the two end points of the different pore radius distribution intervals, where the microscopic physical parameters includes a microscopic pore volume, a microscopic pore frequency, a microscopic porosity, a microscopic tortuosity, a microscopic permeability, a microscopic fluid saturation, and/or a microscopic efficiency of mercury withdrawal of the rock.

According to a third aspect of the present disclosure, there is provided an electronic device, including a processor, and a memory storing executable program instructions thereon which, when executed by the processor, cause the electronic device to implement the method as described in the first aspect.

According to a fourth aspect of the present disclosure, there is provided a non-transitory computer-readable storage medium for determining rock microscopic physical parameters, wherein the computer-readable storage medium has a computer program stored thereon which, when executed by one or more processors, cause the one or more processors to implement the method as described in the first aspect.

From the above description of the aspects, it will be appreciated by those skilled in the art that the present disclosure innovatively provides a method, an apparatus and a computer-readable storage medium for determining rock microscopic physical parameters. The method, the apparatus and the computer-readable storage medium can determine, based on the acquired macroscopic physical parameters and capillary pressure curve experimental data of the rock, an accumulated volume of a nonwetting phase fluid at two end points of different pore radius distribution intervals of the rock, and/or a saturation of the nonwetting phase fluid at the two end points of the different pore radius distribution intervals of the rock, and calculate microscopic physical parameters of the rock, through which the properties of the rock, such as a pore structure, storage, seepage and the like can be depicted, thereby facilitating evaluation of hydrocarbon occurrences of a reservoir and formulation of a reasonable hydrocarbon field development strategy.

### BRIEF DESCRIPTION OF THE DRAWINGS

The above and other objectives, features and advantages of the exemplary embodiments of the present disclosure will become readily apparent from the following detailed description, which proceeds with reference to the accompanying drawings. In the accompanying drawings, several implementations of the present disclosure are illustrated by way of example but not limitation, and like or corresponding reference numerals indicate like or corresponding parts, in which:

FIG. 1 is a schematic flowchart of a method for determining rock microscopic physical parameters according to an embodiment of the present disclosure;

FIG. 2 shows a relation curve of the mercury saturation and the capillary pressure according to an embodiment of the present disclosure;

FIG. 3 shows a first relation curve of the accumulated volume of mercury and the pore radius according to an embodiment of the present disclosure; and

FIG. 4 shows a second relation curve of the mercury saturation and the pore radius according to an embodiment of the present disclosure.

### DETAILED DESCRIPTION

The technical solutions in the embodiments of the present disclosure will be clearly and completely described below with reference to the accompanying drawings in the embodiments of the present disclosure. Obviously, the described embodiments are only some of the embodiments of the present disclosure, but not all of them. All other embodiments, which can be derived by those skilled in the art from the embodiments of the present disclosure without making any creative effort, shall fall within the protection scope of the present disclosure.

According to a first aspect of the present disclosure, there is provided a method for determining rock microscopic physical parameters, through which the determined rock microscopic physical parameters can intuitively characterize properties of microscopic pores in the rock, thereby solving the problem that the physical parameters for characterizing the rock microscopic pore structure in the prior art focus on local or average characteristics of the pore structure, and cannot intuitively reflect the reservoir and seepage characteristics of the rock microscopic pores at both the macroscopic and microscopic levels.

It should be noted here that, on the macroscopic level, a pore in the rock refer to a space not filled with detritus particles, cement or other solid matters, while a thin part connecting pores is called a throat. Further, the porosity of the rock refers to a ratio of a pore volume to an apparent volume of the rock; the permeability of the rock refers to the ability of the rock to allow passage of a fluid under a certain pressure differential; the tortuosity of the rock refers to a ratio of a path length actually covered by fluid particles to an apparent length of the rock when a fluid flows in rock pores; the fluid saturation of the rock refers to is a ratio of a volume occupied by a fluid in rock pores space to a volume of the rock pores; and the efficiency of mercury withdrawal refers to a ratio of a difference between a maximum mercury saturation and a residual mercury saturation while a minimum injection pressure is reduced to a minimal pressure to a maximum mercury saturation.

The pore structure of the rock is a general term for all pore structure features, including forms, sizes, distribution, interrelation of pores and throats in the rock, as well as combinations of channels between pores. To further describe the properties of the microscopic pores in the rock, on the microscopic level, a microscopic pore in the rock refer to a pore in a certain pore radius distribution interval in the rock; the microscopic pore volume of the rock refers to a volume of a certain microscopic pore in the rock, and is called “microscopic pore volume” for short; the microscopic pore frequency of the rock refers to a ratio of a volume of a certain microscopic pore in the rock to a volume of pores in the rock, and is called “microscopic pore frequency” for short; the porosity of a microscopic pore in the rock refers to a ratio of a volume of a certain microscopic pore in the rock to an apparent volume in the rock, and is called “microscopic porosity” for short; the tortuosity of a microscopic pore in the rock refers to a ratio of a path length actually covered by fluid particles to an apparent length of the rock when a fluid flows in a certain microscopic pore of the rock, and is called “microscopic tortuosity” for short; the permeability of a microscopic pore in the rock refers to the ability of a certain microscopic pore in the rock to allow passage of a fluid under a certain pressure differential, and is called “microscopic permeability” for short; the fluid saturation of a microscopic pore in the rock refers to a ratio of a volume occupied by a certain fluid in a certain microscopic pore in the rock to a volume of the microscopic pore, and is called “microscopic fluid saturation” for short; and the efficiency of mercury withdrawal of a microscopic pore in the rock refers to a ratio of a volume of mercury withdrawn from a certain microscopic pore while an injection pressure of the pore structure is detected to be reduced from a maximum value to a minimum value by a mercury intrusion method, to a volume of mercury injected at the same pressure range, and is called “microscopic efficiency of mercury withdrawal” for short.

For the rock mentioned in the above description of the present application, the rock may be, for example, clastic rock, carbonate rock, volcanic rock, crystalline bedrock, argillaceous rock, or any other rock that is suitable for the above method. The specific type of the rock is not limited herein, and those skilled in the art may select it appropriately according to actual needs. It will be appreciated by those skilled in the art that the above fluid is a fluid present in or injected into the rock, including one or more of gas, oil, water or mercury.

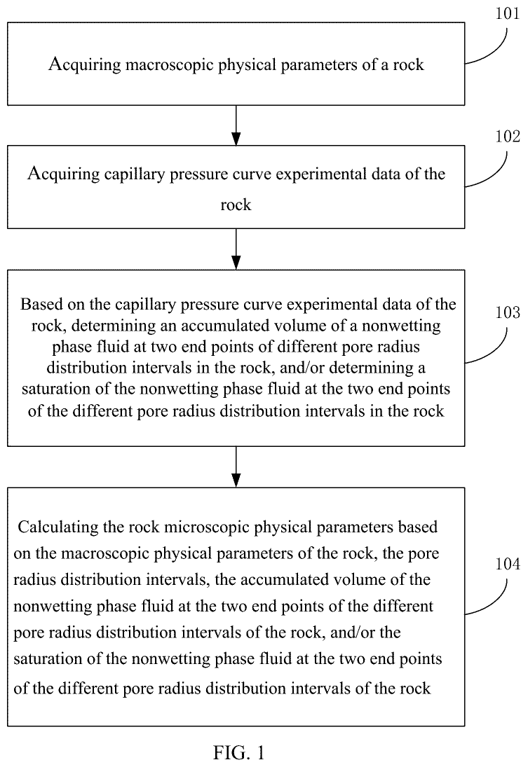

As shown in FIG. 1, the method for determining rock microscopic physical parameters according to an embodiment of the present disclosure includes the following steps S101 to S104.

The step S101 includes: acquiring macroscopic physical parameters of a rock, where the macroscopic physical parameters include an apparent volume, a pore volume, a porosity, a permeability and a fluid saturation of the rock. Further, it will be appreciated by those skilled in the art that the selected rock may be a representative or particularly significant rock in a hydrocarbon reservoir. Illustratively, a rock sample may be selected according to the stratigraphic horizon or depth, or according to the appearance, structure, or other conditions of the rock; a person skilled in the art may select a suitable rock sample according to the actual requirement, which is not limited herein.

The step S102 includes: acquiring capillary pressure curve experimental data of the rock, where the capillary pressure curve experimental data includes a capillary pressure, an accumulated volume of a nonwetting phase fluid, and/or a saturation of a nonwetting phase fluid. Specifically, the selected capillary pressure curve experimental method may be a mercury intrusion method, a semi-permeable barrier method, a centrifuge method or the like. The mercury intrusion method is preferable in this embodiment, because it has the advantages of mature and reliable technology, low requirement on the rock, fast measurement, high measuring pressure and the like, and is particularly suitable for measuring capillary pressure curve experimental data of the rock.

The step S103 includes: based on the capillary pressure curve experimental data of the rock, determining an accumulated volume of a nonwetting phase fluid at two end points of different pore radius distribution intervals, and/or determining a saturation of the nonwetting phase fluid at the two end points of the different pore radius distribution intervals. As one example, this step may specifically include: according to the capillary pressure curve experimental data, making a capillary pressure graph to determine a displacement pressure; according to the displacement pressure, correcting an accumulated volume of a nonwetting phase fluid in the rock, and/or correcting a saturation of the nonwetting phase fluid in the rock, and calculating a pore radius of the rock according to the capillary pressure of the rock; determining a pore radius distribution interval according to radius sizes of pores in the rock; plotting a relation curve of the accumulated volume of the nonwetting phase fluid and the pore radius in a coordinate plot, and determining, according to the determined pore radius distribution interval, volumes of the nonwetting phase fluid at two end points of different pore radius distribution intervals from the curve, and/or plotting a relation curve of the saturation of the nonwetting phase fluid and the pore radius in a coordinate plot, and determining, according to the determined pore radius distribution interval, saturations of the nonwetting phase fluid at two end points of different pore radius distribution intervals from the curve.

It should be noted here that when the capillary pressure curve experimental method for the rock adopts a mercury intrusion method, the nonwetting phase fluid is mercury; when the capillary pressure curve experimental method for the rock adopts a semi-permeable barrier method, the nonwetting phase fluid is vacuum; and when the capillary pressure curve experimental method for the rock adopts a centrifuge method, the nonwetting phase fluid is oil when oil floods water, and the nonwetting phase fluid is water when water floods oil.

Further, the pore radius distribution interval may be a continuous distribution interval, which may be specifically divided into: 0<r≤0.01 μm, 0.01 μm<<r≤0.016 μm, 0.016 μm<r≤0.025 μm, 0.025 μm<r≤0.04 μm, 0.04 μm<r≤0.063 μm, 0.063 μm<r≤0.08 μm, 0.08 μm<r≤0.1 μm, 0.1 μm<r≤0.16 μm, 0.16 μm<r≤0.25 μm, 0.25 μm<r≤0.4 μm, 0.4 μm<r≤0.63 μm, 0.63 μm<r≤ 0.8 μm, 0.8 μm<r≤1 μm, 1 μm<r≤1.6 μm, 1.6 μm<r≤2.5 μm, 2.5 μm<<4 μm, 4 μm<r≤6.3 μm, 6.3 μm<r≤8 μm, 8 μm<r≤10 μm, 10 μm<r≤16 μm, 16 μm<r≤25 μm, 25 μm<r≤40 μm, 40 μm<r≤63 μm and 63 μm<r; or may be divided into: 0<r≤1 μm, 1 μm<r≤10 μm, 10 μm<r≤20 μm, 20 μm<r≤30 μm, 30 μm<r≤40 μm, 40 μm<r≤50 μm and 50 μm<r, where r is the pore radius of the rock.

In other application scenarios, a person skilled in the art may select a plurality of intervals within a continuous distribution interval as pore radius distribution intervals; and in further application scenarios, a person skilled in the art may define the pore radius distribution interval according to the distribution of main pore radii in the reservoir and the precision of a measuring instrument.

It should be noted here that the method for formulating the capillary pressure curve, the method for determining the displacement pressure, the method for correcting the accumulated volume of the nonwetting phase fluid and/or the saturation of the nonwetting phase fluid in the rock, the method for determining the pore radius distribution interval, and the method for calculating the pore radius from the capillary pressure, all belong to the prior art, are well known to those skilled in the art, or can be mastered by learning, and are not described in detail here.

The step S104 includes: calculating the rock microscopic physical parameters based on the macroscopic physical parameters of the rock, the accumulated volume of the nonwetting phase fluid at the two end points of the different pore radius distribution intervals of the rock, and/or the saturation of the nonwetting phase fluid at the two end points of the different pore radius distribution intervals of the rock, where the microscopic physical parameters includes a microscopic pore volume, a microscopic porosity, a microscopic tortuosity, a microscopic permeability, a microscopic fluid saturation, and/or a microscopic efficiency of mercury withdrawal of the rock, where microscopic pores include microscopic pores with saturated nonwetting phase fluid and microscopic pores with minimum unsaturated nonwetting phase fluid. where calculating the microscopic physical parameters of the rock may include dividing the rock into a plurality of microscopic pores.

It will be appreciated by those skilled in the art that other capillary pressure curve experimental methods, such as the semi-permeable barrier methods, the centrifuge methods, and the like, may also be performed with reference to the method of this embodiment.

As will be understood by those skilled in the art in light of the foregoing description, the embodiment of the present disclosure innovatively provides a method for determining rock microscopic physical parameters, which can determine, based on the acquired macroscopic physical parameters and capillary pressure curve experimental data of the rock, microscopic physical parameters of the rock, through which the properties of the rock, such as a pore structure, storage, seepage and the like can be depicted, thereby facilitating evaluation of hydrocarbon occurrences of a reservoir and formulation of a reasonable hydrocarbon field development strategy.

To facilitate understanding of the following equations, it should be noted that according to the pore radius distribution interval, when the rock is divided into n pore radius distribution intervals, there are n microscopic pores in the rock; on the capillary pressure curve of a saturated nonwetting phase fluid, n end points of the pore radius distribution intervals, n−1 pore radius distribution intervals, and n−1 microscopic pores are present; a maximum injection pressure of the nonwetting phase fluid is reached at the end points of a pore radius rₙ; and when the maximum injection pressure is reached, no nonwetting phase fluid is injected if the radius of the microscopic pore is smaller than rₙ. Such a microscopic pore, which is the n^{th} microscopic pore, is called a microscopic pore of a minimum unsaturated nonwetting phase fluid.

The microscopic pore volume mentioned above may be calculated by any one of the following methods:

As one example, for an i^{th} microscopic pore with saturated nonwetting phase fluid in the rock, the pore volume is calculated by:

$${V}_{P i} = {V}_{H g j ( i + 1 )} - {V}_{H g j i} = Δ {V}_{H g j i} \qquad ( 1 )$$

where: V_{Pi} is the volume of the i^{th} microscopic pore in the rock in the unit of cm³; V_{Hgji} is the accumulated volume of the nonwetting phase fluid in the rock corresponding to one end point value rᵢ of an i^{th} pore radius distribution interval, which may be in the unit of cm³; V_{Hgj(i+1)} is the accumulated volume of the nonwetting phase fluid in the rock corresponding to the other end point value rᵢ₊₁ of the i^{th} pore radius distribution interval, which may be in the unit of cm³; ΔV_{Hgji} is the volume of the nonwetting phase fluid injected into the i^{th} microscopic pore in the rock, which may be in the unit of cm³; n is the total number of microscopic pores divided from the rock according to the pore radius distribution intervals; i is a sequence number of a microscopic pore in the rock, and i∈[1,n−1]; rᵢ and rᵢ₊₁ are pore radii of the rock, which may be in the unit of μm, and rᵢ₊₁<rᵢ.

Further, for a microscopic pore with minimum unsaturated nonwetting phase fluid in the rock, which is an n^{th} microscopic pore, the pore volume is calculated by:

$${V}_{P n} = {V}_{P} - {V}_{H g j n} = {V}_{P} - {\sum }_{1}^{n - 1} {V}_{p i} \qquad ( 2 )$$

where: V_{Pn} is the volume of the n^{th} microscopic pore in the rock, which may be in the unit of cm³; V_{P} is the pore volume of the rock, which may be in the unit of cm³; and V_{Hgjn} is the accumulated volume of the nonwetting phase fluid injected into the rock at a maximum injection pressure, which may be in the unit of cm³.

As another example, for an i^{th} microscopic pore with saturated nonwetting phase fluid in the rock, the pore volume is calculated by:

$${V}_{P i} = ( {S}_{H g j i + 1} - {S}_{H g j i} ) {V}_{P} = Δ {S}_{H g j i} {V}_{P} \qquad ( 3 )$$

where: V_{Pi} is the volume of the i^{th} microscopic pore in the rock; which may be in the unit of cm³; S_{Hgji} is the saturation of the nonwetting phase fluid in the rock corresponding to one end point value rᵢ of the i^{th} pore radius distribution interval; S_{Hgj(i+1)} is the saturation of the nonwetting phase fluid in the rock corresponding to the other end point value rᵢ₊₁ of the i^{th} pore radius distribution interval; ΔS_{Hgji} is the saturation of the nonwetting phase fluid injected into the i^{th} microscopic pore in the rock; V_{P} is the pore volume of the rock, which may be in the unit of cm³; n is the total number of microscopic pores divided from the rock according to the pore radius distribution intervals; i is a sequence number of a microscopic pore, and i∈[1, n−1]; r; and rᵢ₊₁ are pore radii, which may be in the unit of μm, and rᵢ₊₁<rᵢ.

Further, for a microscopic pore with minimum unsaturated nonwetting phase fluid in the rock, which is an n^{th} microscopic pore, the pore volume is calculated by:

$${V}_{P n} = ( 1 - {S}_{H g j n} ) {V}_{P} \qquad ( 4 )$$

where: V_{Pn} is the volume of the n^{th} microscopic pore in the rock, which may be in the unit of cm³; V_{P} is the pore volume of the rock, which may be in the unit of cm³; and S_{Hgjn} is the saturation of the nonwetting phase fluid injected into the rock at a maximum injection pressure.

The microscopic pore frequency mentioned above may be calculated by any one of the following methods:

As one example, for an i^{th} microscopic pore with saturated nonwetting phase fluid in the rock, the pore frequency is calculated by:

$${f}_{i} = \frac{{V}_{H g j ( i + 1 )} - {v}_{H g j i}}{{V}_{P}} = \frac{Δ {V}_{H g j i}}{{V}_{P}} \qquad ( 5 )$$

where: fᵢ is the frequency of the i^{th} microscopic pore in the rock; V_{Hgji} is the accumulated volume of the nonwetting phase fluid in the rock corresponding to one end point value rᵢ of an i^{th} pore radius distribution interval; V_{Hgj(i+1)} is the accumulated volume of the nonwetting phase fluid in the rock corresponding to the other end point value rᵢ₊₁ of the i^{th} pore radius distribution interval; ΔV_{Hgji} is the volume of the nonwetting phase fluid injected into the i^{th} microscopic pore in the rock; V_{P} is the pore volume of the rock, which may be in the unit of cm³; n is the total number of microscopic pores divided from the rock according to the pore radius distribution intervals; and i is a sequence number of a microscopic pore, and i∈[1, n−1]; and rᵢ and rᵢ₊₁ are pore radii, and rᵢ₊₁<rᵢ.

Further, for a microscopic pore with minimum unsaturated nonwetting phase fluid in the rock, which is an n^{th} microscopic pore, the pore frequency is calculated by:

$${f}_{n} = \frac{{V}_{P} - {V}_{H g j n}}{{V}_{P}} \qquad ( 6 )$$

where: fₙ is the frequency of the n^{th} microscopic pore in the rock; V_{P} is the pore volume of the rock, which may be in the unit of cm³; and V_{Hgjn} is the accumulated volume of the nonwetting phase fluid injected into the rock at a maximum injection pressure, which may be in the unit of cm³.

As another example, for an i^{th} microscopic pore with saturated nonwetting phase fluid in the rock, the pore frequency is calculated by:

$${f}_{i} = {S}_{H g j ( i + 1 )} - {S}_{H g j i} = Δ {S}_{H g j i} \qquad ( 7 )$$

where: fᵢ is the frequency of the i^{th} microscopic pore in the rock; S_{Hgji} is the saturation of the nonwetting phase fluid injected into the rock corresponding to one end point value rᵢ of the i^{th} pore radius distribution interval; S_{Hgj(i+1)} is the saturation of the nonwetting phase fluid injected into the rock corresponding to the other end point value rᵢ₊₁ of the i^{th} pore radius distribution interval; ΔS_{Hgji} is the saturation of the nonwetting phase fluid injected into the i^{th} microscopic pore in the rock; n is the total number of microscopic pores of divided from the rock according to the pore radius distribution intervals; and i is a sequence number of a microscopic pore, and i ∈[1, n−1]. and rᵢ and rᵢ₊₁ are pore radii, and rᵢ₊₁<rᵢ;
Further, for the minimum unsaturated microscopic pore in the rock, which is an n^{th} microscopic pore, the pore frequency is calculated by:

$${f}_{n} = 1 - {S}_{H g j n} = 1 - {\sum }_{1}^{n - 1} {f}_{i} \qquad ( 8 )$$

where: fₙ is the frequency of the n^{th} microscopic pore in the rock; and S_{Hgjn} is the saturation of the nonwetting phase fluid injected into the rock at a maximum injection pressure.

The microscopic porosity mentioned above may be calculated by any one of the following methods:

As one example, for an i^{th} microscopic pore with saturated nonwetting phase fluid in the rock, the porosity is calculated by:

$${∅}_{i} = \frac{{V}_{H g j ( i + 1 )} - {V}_{H g j i}}{{V}_{b}} = \frac{Δ {V}_{H g j i}}{{v}_{b}} \qquad ( 9 )$$

where: Øᵢ is the porosity of the i^{th} microscopic pore in the rock; V_{P} is the apparent volume of the rock; which may be in the unit of cm³; V_{Hgji} is the accumulated volume of the nonwetting phase fluid in the rock corresponding to one end point value rᵢ of an i^{th} pore radius distribution interval; V_{Hej(i+1)} is the accumulated volume of the nonwetting phase fluid in the rock corresponding to the other end point value rᵢ₊₁ of the i^{th} pore radius distribution interval; ΔV_{Hgji} is the volume of the nonwetting phase fluid injected into the i^{th} microscopic pore in the rock, which may be in the unit of cm³; n is the total number of microscopic pores divided from the rock according to the pore radius distribution intervals; and i is a sequence number of a microscopic pore, and i∈[1, n−1]; and rᵢ and rᵢ₊₁ are pore radii, and rᵢ₊₁<rᵢ.

Further, for the minimum unsaturated microscopic pore in the rock, which is an n^{th} microscopic pore, the porosity is calculated by:

$${∅}_{n} = \frac{{V}_{b} - {V}_{H g j n}}{{V}_{b}} \qquad ( 10 )$$

where: Øₙ is the porosity of the n^{th} microscopic pore in the rock; V_{P} is the apparent volume of the rock, which may be in the unit of cm³; and V_{Hgjn} is the accumulated volume of the nonwetting phase fluid injected into the rock at a maximum injection pressure, which may be in the unit of cm³.

As another example, for an i^{th} microscopic pore with saturated nonwetting phase fluid in the rock, the porosity is calculated by:

$${∅}_{i} = ( {S}_{H g j ( i + 1 )} - {S}_{H g j i} ) ∅ = Δ {S}_{H g j i} ∅ \qquad ( 11 )$$

where: Øᵢ is the porosity of the i^{th} microscopic pore in the rock; Ø is the porosity of the rock; S_{Hgji} is the saturation of the nonwetting phase fluid injected into the rock corresponding to one end point value rᵢ of the i^{th} pore radius distribution interval; S_{Hgj(i+1)} is the saturation of the nonwetting phase fluid injected into the rock corresponding to the other end point value rᵢ₊₁ of the i^{th} pore radius distribution interval; ΔS_{Hgji} is the saturation of the nonwetting phase fluid injected into the i^{th} microscopic pore in the rock; n is the total number of microscopic pores divided from the rock according to the pore radius distribution intervals; and i is a sequence number of a microscopic pore, and i∈[1, n−1]; r; and rᵢ₊₁ are pore radii, and rᵢ₊₁<rᵢ.

Further, for a microscopic pore with the minimum unsaturated nonwetting phase fluid in the rock, which is an n^{th} microscopic pore, the porosity is calculated by:

$${∅}_{n} = ( 1 - {S}_{H g j n} ) ∅ = ∅ - {Σ}_{1}^{n - 1} {∅}_{i} \qquad ( 12 )$$

where: Øₙ is the porosity of the n^{th} microscopic pore in the rock; Ø is the porosity of the rock; Øᵢ is the porosity of the i^{th} microscopic pore in the rock; and S_{Hgjn} is the saturation of the nonwetting phase fluid injected into the rock at a maximum injection pressure, where i is a sequence number, and i∈[1, n−1].
as another example, the porosity of an i^{th} microscopic pore in the rock is calculated by:

$${∅}_{i} = {f}_{i} ∅ \qquad ( 13 )$$

where: Øᵢ is the porosity of the i^{th} microscopic pore in the rock; fᵢ is the frequency of the i^{th} microscopic pore in the rock; Ø is the porosity of the rock; n is the total number of microscopic pores divided from the rock according to the pore radius distribution intervals; and i is a sequence number of a microscopic pore, and i∈[1,n].

The microscopic tortuosity mentioned above may be calculated by any one of the following methods:

As one example, the tortuosity of the i^{th} microscopic pore in the rock, when neglecting the influence of the minimum unsaturated microscopic pore, is calculated by:

$${τ}_{i} = \sqrt{1 + 2 ( 1 - \frac{( n - 1 ) {∅}_{i}}{{Σ}_{1}^{n - 1} {∅}_{i}} ∅ )} \qquad ( 14 )$$

where: τᵢ is the tortuosity of the i^{th} microscopic pore in the rock; Ø is the porosity of the rock; Øᵢ is the porosity of the i^{th} microscopic pore in the rock; n is the total number of microscopic pores divided from the rock according to the pore radius distribution intervals; and i is a sequence number of a microscopic pore, and i∈[1, n−1].

As another example, the tortuosity of the i^{th} microscopic pore in the rock, when neglecting the influence of the minimum unsaturated microscopic pore, is calculated by:

$${τ}_{i} = 1 + α ( 1 - \frac{( n - 1 ) {∅}_{i}}{{Σ}_{1}^{n - 1} {∅}_{i}} ∅ ) \qquad ( 15 )$$

where: τᵢ is the tortuosity of the i^{th} microscopic pore in the rock; a is an equation coefficient which may be 0.75; Ø is the porosity of the rock; Øᵢ is the porosity of the i^{th} microscopic pore in the rock; n is the total number of microscopic pores divided from the rock according to the pore radius distribution intervals; and i is a sequence number, and i ∈[1, n−1].

The microscopic permeability mentioned above may be calculated by any one of the following methods:

As one example, the permeability of the i^{th} microscopic pore in the rock, when neglecting the influence of the minimum unsaturated microscopic pore, is calculated by any one of the following equations (16) to (18):

$${k}_{i} = \frac{{( {r}_{i} + {r}_{i + 1} )}^{2} \frac{{∅}_{i}}{{τ}_{i}^{2}}}{{Σ}_{1}^{n - 1} {( {r}_{i} + {r}_{i + 1} )}^{2} \frac{{∅}_{i}}{{τ}_{i}^{2}}} K \qquad ( 16 )$$

$${k}_{i} = \frac{{( {r}_{i} + {r}_{i + 1} )}^{2} \frac{{∅}_{i}}{{τ}_{j}}}{{Σ}_{1}^{n - 1} {( {r}_{i} + {r}_{i + 1} )}^{2} \frac{{∅}_{j}}{{τ}_{j}}} K \qquad ( 17 )$$

$${k}_{i} = \frac{{( {r}_{i} + {r}_{i + 1} )}^{2} {∅}_{i}}{{Σ}_{1}^{n - 1} {( {r}_{i} + {r}_{i + 1} )}^{2} {∅}_{i}} K \qquad ( 18 )$$

where: k; is the permeability the i^{th} microscopic pore in the rock, which may be in the unit of μm²; Øᵢ is the porosity of the i^{th} microscopic pore in the rock; r; is the pore radius at one end point of an i^{th} pore radius distribution interval, which may be in the unit of μm; rᵢ₊₁ is the pore radius at the other end point of the i^{th} pore radius distribution interval, which may be in the unit of μm; Ti is the tortuosity of the i^{th} microscopic pore in the rock, which is dimensionless; K is the permeability of the rock, which may be in the unit of μm²; n is the total number of microscopic pores of divided from the rock according to the pore radius distribution intervals; and i is a sequence number of a microscopic pore, and i∈[1, n−1].

The microscopic fluid saturation mentioned above may be calculated by:
acquiring measurement results of the porosity, the fluid saturation, the microscopic porosity or the microscopic pore frequency of the rock;
sequencing the microscopic pores in the rock according to sizes of pore radii;
obtaining filling sequences and filling capacities of different fluids in different pore throats; and
preliminarily determining a value of the microscopic fluid saturation based on the filling sequences and the filling capacities of different fluids in different pore throats. Further, as one example: when the rock contains three phases, water, oil and water, the microscopic pore with the largest pore radius is firstly filled with gas, that is, the microscopic gas saturation is 1; the microscopic pore with a larger pore radius is preferably filled with gas, then with gas and oil, then with oil again, and then with oil and water; the microscopic pore with a smaller pore radius is preferably filled with oil, then with oil and water, and then with water; and the microscopic pore with the smallest pore radius is completely filled with water, that is, the microscopic water saturation is 1. When the rock contains two phases, oil and water, the microscopic pore with the largest pore radius is filled with oil, that is, the microscopic oil saturation is 1; the microscopic pore with a larger pore radius is preferably filled with oil, then with oil and water, and then with water; and the microscopic pore with the smallest pore radius is completely filled with water, that is, the microscopic water saturation is 1. When the rock contains two phases, gas and water, the microscopic pore with the largest pore radius is filled with gas, that is, the microscopic gas saturation is 1; the microscopic pore with a larger pore radius is preferably filled with gas, then with gas and water, and then with water; and the microscopic pore with the smallest pore radius is completely filled with water, that is, the microscopic water saturation is 1.

A preliminary discrimination result of each microscopic fluid saturation in the rock is substituted into relational expressions (19), (20) and (21) of the fluid saturation, the porosity, the microscopic fluid saturation and the microscopic porosity; or a preliminary discrimination result of each microscopic fluid saturation in the rock is substituted into relational expressions (22), (23) and (24) of the fluid saturation, the microscopic fluid saturation and the microscopic pore frequency. Then, distribution of fluid saturations in the microscopic pores can be obtained by simultaneous solution and continuous trial calculation.

$${{\sum }^{n}}_{1} {∅}_{i} {S}_{g i} = ∅ {S}_{g} \qquad ( 19 )$$

$${{\sum }^{n}}_{1} {∅}_{i} {S}_{o i} = ∅ {S}_{o} \qquad ( 20 )$$

$${{\sum }^{n}}_{1} {∅}_{i} {S}_{w i} = ∅ {S}_{w} \qquad ( 21 )$$

$${{\sum }^{n}}_{1} {f}_{i} {S}_{g i} = {S}_{g} \qquad ( 22 )$$

$${{\sum }^{n}}_{1} {f}_{i} {S}_{o i} = {S}_{o} \qquad ( 23 )$$

$${{\sum }^{n}}_{1} {f}_{i} {S}_{w i} = {S}_{w} \qquad ( 24 )$$

where: Sₒᵢ is the oil saturation of the i^{th} microscopic pore in the rock; Sₒ is the oil saturation of the rock; S_{wi} is the water saturation of the i^{th} microscopic pore in the rock; S_{w} is the water saturation of the rock; S_{gi} is the gas saturation of the i^{th} microscopic pore in the rock; Sg is the gas saturation of the rock; fᵢ is the frequency of the i^{th} microscopic pore in the rock; Øᵢ is the porosity of the i^{th} microscopic pore in the rock; Ø is the porosity the rock; i is a sequence number of a microscopic pore, and i∈[1,n]; and n is the total number of microscopic pores divided from the rock according to the pore radius distribution intervals.

The microscopic efficiency of mercury withdrawal mentioned above may be calculated by any one of the following methods:

As one example, the efficiency of mercury withdrawal of an i^{th} microscopic pore in the rock is calculated by:

$${W}_{E i} = \frac{{V}_{H g t ( i + 1 )} - {V}_{H g t i}}{{V}_{H g j ( i + 1 )} - {V}_{H g j i}} = \frac{Δ {V}_{H g t i}}{Δ {V}_{H g j i}} \qquad ( 25 )$$

where: W_{Ei} is the efficiency of mercury withdrawal of the i^{th} microscopic pore in the rock; V_{Hgji} is the accumulated volume of mercury in the rock corresponding to one end point rᵢ of the i^{th} pore radius distribution interval in a mercury feeding process, which may be in the unit of cm³; V_{Hgj(i+1)} is the accumulated volume of mercury in the rock corresponding to the other end point rᵢ₊₁ of the i^{th} pore radius distribution interval in the mercury feeding process, which may be in the unit of cm³; V_{Hgti} is the accumulated volume of mercury in the rock corresponding to one end point rᵢ of the i^{th} pore radius distribution interval in a mercury withdrawal process, which may be in the unit of cm³; V_{Hgt(i+1)} is the accumulated volume of mercury in the rock corresponding to the other end point rᵢ₊₁ of the i^{th} pore radius distribution interval in the rock in the mercury withdrawal process, which may be in the unit of cm³; ΔV_{Hgji} is the volume of mercury injected into the i^{th} microscopic pore in the rock in the mercury feeding process, which may be in the unit of cm³; ΔV_{Hgti} is the volume of mercury withdrawn from the i^{th} microscopic pore in the rock in the mercury withdrawal process, which may be in the unit of cm³; n is the total number of microscopic pores divided from the rock according to the pore radius distribution intervals; and i is a sequence number of a microscopic pore in the rock, and i ∈[1,n−1]; rᵢ and rᵢ₊₁ are pore radii of the rock, which may be in the unit of μm, and rᵢ₊₁<rᵢ.

As another example, the efficiency of mercury withdrawal of an i^{th} microscopic pore in the rock is calculated by:

$${W}_{E i} = \frac{{S}_{H g t ( i + 1 )} - {S}_{H g t i}}{{S}_{H g j ( i + 1 )} - {S}_{H g j i}} = \frac{Δ {S}_{H g t i}}{Δ {S}_{H g j i}} \qquad ( 26 )$$

where: W_{Ei} is the efficiency of mercury withdrawal of the i^{th} microscopic pore in the rock; S_{Hgji} is the mercury saturation in the rock corresponding to one end point rᵢ of the i^{th} pore radius distribution interval in a mercury feeding process, and S_{Hgji} is a decimal; S_{Hgj(i+1)} is the mercury saturation in the rock corresponding to the other end point rᵢ₊₁ of the i^{th} pore radius distribution interval in the mercury feeding process; S_{Hgti} is the mercury saturation in the rock corresponding to one end point rᵢ of the i^{th} pore radius distribution interval in a mercury withdrawal process; S_{Hgt(i+1)} is the mercury saturation in the rock corresponding to the other end point rᵢ₊₁ of the i^{th} pore radius distribution interval in the mercury withdrawal process; ΔS_{Hgji} is the saturation of mercury injected into the i^{th} microscopic pore in the rock in the mercury feeding process; ΔS_{Hgti} is the saturation of mercury withdrawn from the i^{th} microscopic pore in the rock in the mercury withdrawal process; n is the total number of microscopic pores of divided from the rock according to the pore radius distribution intervals; and i is a sequence number of a microscopic pore in the rock, and i∈[1, n−1]; r and rᵢ₊₁ are pore radii of the rock, which may be in the unit of μm, and rᵢ₊₁<rᵢ.

In the rock capillary pressure curve experiment, two fluids, a wetting phase fluid and a nonwetting phase fluid, are present in rock pores, a summed saturation of the wetting phase fluid and the nonwetting phase fluid is 1, and a summed accumulated volume of the wetting phase fluid and the nonwetting phase fluid is equal to the pore volume. For convenience of description, the above equations use the saturation of the nonwetting phase fluid and the accumulated volume of the nonwetting phase fluid, while other equation can be obtained by simple substitutions if the saturation of the wetting phase fluid and accumulated volume of the wetting phase fluid are used.

According to a second aspect of the present disclosure, there is provided an apparatus for determining rock microscopic physical parameters, including: a first acquisition unit, a second acquisition unit, a third acquisition unit and a logical computation unit.

The first acquisition unit is configured to acquire macroscopic physical parameters of a rock, where the macroscopic physical parameters includes an apparent volume, a pore volume, a porosity, a permeability and/or a fluid saturation of the rock. The second acquisition unit is configured to acquire capillary pressure curve experimental data of the rock, where the experimental data includes a capillary pressure, an accumulated volume of a nonwetting phase fluid, and/or a saturation of a nonwetting phase fluid. The third acquisition unit is configured to acquire an accumulated volume of a nonwetting phase fluid at two end points of different pore radius distribution intervals of the rock, and/or a saturation of the nonwetting phase fluid at the two end points of the different pore radius distribution intervals of the rock. The logical computation unit is configured to calculate the rock microscopic physical parameters based on the macroscopic physical parameters of the rock, the accumulated volume of the nonwetting phase fluid at the two end points of the different pore radius distribution intervals of the rock, and/or the saturation of the nonwetting phase fluid at the two end points of the different pore radius distribution intervals of the rock, where the microscopic physical parameters includes a microscopic pore volume, a microscopic pore frequency, a microscopic porosity, a microscopic tortuosity, a microscopic permeability, a microscopic fluid saturation, and/or a microscopic efficiency of mercury withdrawal.

According to a third aspect of the present disclosure, there is provided an electronic device, including a processor, and a memory storing executable program instructions thereon which, when executed by the processor, cause the electronic device to implement part or all steps of the method according to the present disclosure.

The processor may control operation of the electronic device. For example, the processor controls operation of the electronic device by executing a program stored on memory in the electronic device. The processor may be implemented by a central processing unit (CPU), an application processor (AP), an intelligent processing unit (IPU), or the like provided in the electronic device. However, the present disclosure is not limited thereto. In this implementation, the processor may be implemented in any suitable manner. For example, the processor may take the form of, for example, a microprocessor or processor and a computer-readable medium that stores computer-readable program codes (e.g., software or firmware) executable by the (micro) processor, a logic gate, a switch, an application specific integrated circuit (ASIC), a programmable logic controller, an embedded microcontroller, or the like.

The memory may be used for hardware to store various data and instructions processed in the electronic device. For example, the memory may store processed data and data to be processed in the electronic device. The memory may store a processed data set or a data set to be processed by the processor. Further, the memory may store applications to be driven by the electronic device, drivers, and the like. The memory may be a DRAM, but the present disclosure is not limited thereto. The memory may include at least one of a volatile memory or a non-volatile memory. The non-volatile memory may include a read-only memory (ROM), a programmable ROM (PROM), an electrically programmable ROM (EPROM), an electrically erasable programmable ROM (EEPROM), a flash, phase-change RAM (PRAM), a magnetic RAM (MRAM), a resistive RAM (RRAM), a ferroelectric RAM (FRAM), or the like. The volatile memory may include a dynamic RAM (DRAM), a static RAM (SRAM), a synchronous DRAM (SDRAM), a PRAM, an MRAM, an RRAM, a ferroelectric RAM (FeRAM), or the like. In one embodiment, the memory may include at least one of a hard disk drive (HDD), a solid state drive (SSD), a high density flash memory (CF), a secure digital (SD) card, a micro-SD card, a Mini-SD card, an XD card, a cache, or a memory stick.

To sum up, specific functions implemented by the memory and the processor of the electronic device provided in the implementation of the present disclosure may be explained in comparison with the foregoing implementations in the description of the present disclosure, and can achieve the technical effects of the foregoing implementations, and therefore, no further description is provided herein.

According to a fourth aspect of the present disclosure, there is provided a computer-readable storage medium (or a non-transitory machine-readable storage medium, or a machine-readable storage medium) having stored thereon computer program instructions (or a computer program, or computer instruction codes) which, when executed by one or more processors of an electronic device (or an electronic server, or the like), cause the one or more processors to implement part or all steps of the method according to the present disclosure.

It should be noted that although in the above detailed description several means or sub-means of the electronic device are mentioned, this division is not mandatory. Indeed, according to implementations of the present disclosure, the features and functions of two or more of the devices described above may be embodied in one device. Conversely, the features and functions of one device described above may be further divided to be embodied in a plurality of devices.

To facilitate further understanding of the technical solutions of the present application by those skilled in the art, the method for determining rock microscopic physical parameters discussed above will be described in detail below with reference to specific embodiments and accompanying drawings.

Illustratively, a selected rock in a target reservoir interval of a target oilfield is used as an example for the description below. To calculate the microscopic volume of mercury withdrawal, a mercury intrusion method is used in the capillary pressure curve experiment.

Macroscopic physical parameters of the rock are acquired and shown in table 1. In the data obtained, the rock apparent volume, the pore volume, the air permeability, and the porosity are actually measured data, while the fluid saturation is artificially given.

TABLE 1

| | |
|---|---|
| Rock apparent volume (cm³) | 12.612 |
| Pore volume (cm³) | 2.259 |
| Air permeability (10⁻³ μm²) | 3.70 |
| Porosity (%) | 0.185 |
| Gas saturation | 0.24 |
| Oil saturation | 0.46 |
| Water saturation | 0.30 |

Capillary pressure curve experimental data of the rock is acquired, and shown in table 2.

TABLE 2

| | | | | Mercury | Mercury |
|---|---|---|---|---|---|
| | | Volume of | Volume of | saturation | saturation |
| | | injected | withdrawn | of injected | of withdrawn |
| | Capillary | mercury | mercury | mercury | mercury |
| | pressure | V_{Hgji} | V_{Hgti} | S_{Hgji} | S_{Hgti} |
| No. | (MPa) | (cm³) | (cm³) | (decimal) | (decimal) |
| 1 | 0.00753 | 0.000 | 0.000 | 0.00000 | 0.00000 |
| 2 | 0.02171 | 0.015 | 0.000 | 0.00651 | 0.00000 |
| 3 | 0.03543 | 0.022 | 0.000 | 0.00957 | 0.00000 |
| 4 | 0.04910 | 0.025 | 0.000 | 0.01128 | 0.00000 |
| 5 | 0.06274 | 0.029 | 0.000 | 0.01266 | 0.00000 |
| 6 | 0.07634 | 0.031 | 0.000 | 0.01369 | 0.00000 |
| 7 | 0.09705 | 0.035 | 0.000 | 0.01554 | 0.00000 |
| 8 | 0.11754 | 0.037 | 0.000 | 0.01660 | 0.00000 |
| 9 | 0.13815 | 0.041 | 1.697 | 0.01813 | 0.75118 |
| 10 | 0.20671 | 0.047 | 1.713 | 0.02067 | 0.75835 |
| 11 | 0.34500 | 0.071 | 1.748 | 0.03132 | 0.77377 |
| 12 | 0.48159 | 0.090 | 1.779 | 0.03984 | 0.78744 |
| 13 | 0.69430 | 0.123 | 1.810 | 0.05426 | 0.80143 |
| 14 | 1.03204 | 0.216 | 1.840 | 0.09563 | 0.81463 |
| 15 | 1.37738 | 0.344 | 1.858 | 0.15230 | 0.82252 |
| 16 | 1.71762 | 0.471 | 1.872 | 0.20872 | 0.82865 |
| 17 | 2.07177 | 0.602 | 1.883 | 0.26635 | 0.83343 |
| 18 | 2.74879 | 0.812 | 1.898 | 0.35943 | 0.84039 |
| 19 | 4.15954 | 1.119 | 1.920 | 0.49524 | 0.85006 |
| 20 | 5.49350 | 1.318 | 1.934 | 0.58324 | 0.85598 |
| 21 | 6.86818 | 1.467 | 1.945 | 0.64929 | 0.86112 |
| 22 | 8.28656 | 1.566 | 1.955 | 0.69316 | 0.86540 |
| 23 | 10.41610 | 1.653 | 1.966 | 0.73175 | 0.87044 |
| 24 | 12.44678 | 1.706 | 1.974 | 0.75518 | 0.87395 |
| 25 | 15.85773 | 1.770 | 1.984 | 0.78367 | 0.87842 |
| 26 | 20.61262 | 1.840 | 1.992 | 0.81460 | 0.88166 |
| 27 | 24.10652 | 1.876 | 1.995 | 0.83026 | 0.88296 |
| 28 | 27.50519 | 1.906 | 1.996 | 0.84393 | 0.88374 |
| 29 | 34.42876 | 1.958 | 1.998 | 0.86694 | 0.88449 |
| 30 | 41.30549 | 1.999 | 1.999 | 0.88499 | 0.88499 |

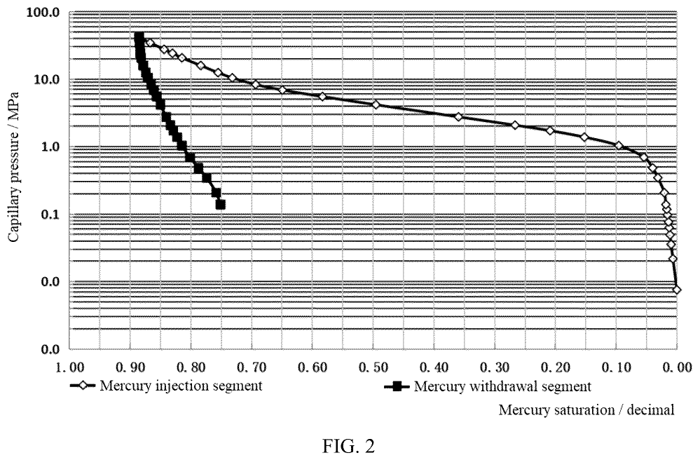

Based on the capillary pressure curve experimental data of the rock, an accumulated volume of a nonwetting phase fluid at two end points of different pore radius distribution intervals is determined, and/or a saturation of the nonwetting phase fluid at the two end points of the different pore radius distribution intervals is determined. Specifically, a capillary pressure curve is plotted according to the capillary pressure experimental data (FIG. 2), and a displacement pressure is determined to be 0.69430 MPa. The accumulated volume and saturation of mercury are corrected according to the displacement pressure, and a pore radius is calculated from the capillary pressure. The 5 obtained data is shown in table 3.

TABLE 3

| | | | | | Mercury | Mercury |
|---|---|---|---|---|---|---|
| | | | Volume of | Volume of | saturation of | saturation of |
| | | | injected | withdrawn | injected | withdrawn |
| | Capillary | Pore | mercury | mercury | mercury | mercury |
| | pressure | radius | V_{Hgji} | V_{Hgti} | S_{Hgji} | S_{Hgti} |
| No. | (MPa) | (μm) | (cm³) | (cm³) | (decimal) | (decimal) |
| 1 | 0.6943 | 1.05861 | 0 | 1.688 | 0 | 0.74717 |
| 2 | 1.03204 | 0.71218 | 0.093 | 1.718 | 0.04137 | 0.76037 |
| 3 | 1.37738 | 0.53362 | 0.221 | 1.736 | 0.09804 | 0.76826 |
| 4 | 1.71762 | 0.42792 | 0.349 | 1.749 | 0.15446 | 0.77439 |
| 5 | 2.07177 | 0.35477 | 0.479 | 1.760 | 0.21209 | 0.77917 |
| 6 | 2.74879 | 0.26739 | 0.689 | 1.776 | 0.30517 | 0.78613 |
| 7 | 4.15954 | 0.17670 | 0.996 | 1.798 | 0.44098 | 0.79579 |
| 8 | 5.4935 | 0.13380 | 1.195 | 1.811 | 0.52898 | 0.80172 |
| 9 | 6.86818 | 0.10702 | 1.344 | 1.823 | 0.59503 | 0.80686 |
| 10 | 8.28656 | 0.08870 | 1.443 | 1.832 | 0.63889 | 0.81114 |
| 11 | 10.4161 | 0.07056 | 1.530 | 1.844 | 0.67749 | 0.81618 |
| 12 | 12.44678 | 0.05905 | 1.583 | 1.852 | 0.70092 | 0.81969 |
| 13 | 15.85773 | 0.04635 | 1.648 | 1.862 | 0.72941 | 0.82416 |
| 14 | 20.61262 | 0.03566 | 1.718 | 1.869 | 0.76034 | 0.8274 |
| 15 | 24.10652 | 0.03049 | 1.753 | 1.872 | 0.77600 | 0.8287 |
| 16 | 27.50519 | 0.02672 | 1.784 | 1.874 | 0.78967 | 0.82948 |
| 17 | 34.42876 | 0.02135 | 1.836 | 1.876 | 0.81268 | 0.83023 |
| 18 | 41.30549 | 0.01779 | 1.877 | 1.877 | 0.83073 | 0.83073 |

According to the pore radius, the pore radius distribution intervals are determined to be: 0<rᵢ≤0.016 μm, 0.016 μm<rᵢ≤0.025 μm, 0.025 μm<rᵢ≤0.040 μm, 0.040 μm<rᵢ≤0.063 μm, 0.063 μm<rᵢ≤0.08 μm, 0.08 μm<rᵢ≤0.1 μm, 0.1 μm<rᵢ≤0.16 μm, 0.16 μm<rᵢ≤0.25 μm, 0.25 μm<rᵢ≤0.40 μm, 0.40 μm<rᵢ≤0.63 μm, 0.63 μm<rᵢ≤0.80 μm, 0.80 μm<rᵢ≤1.00 μm and 1.00 μm<rᵢ≤1.60 μm.

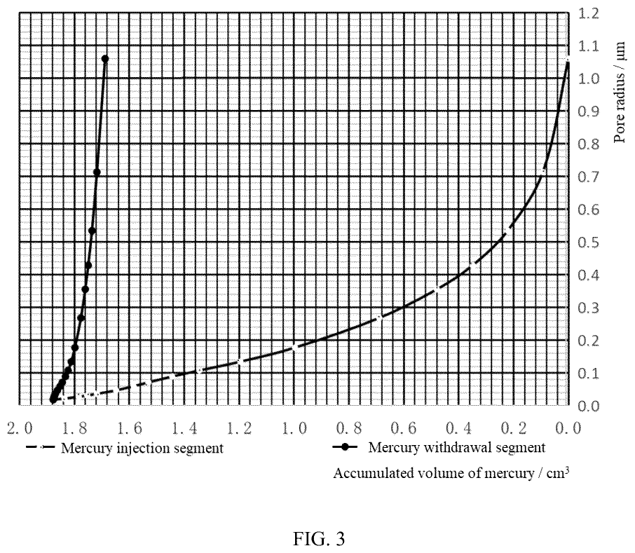

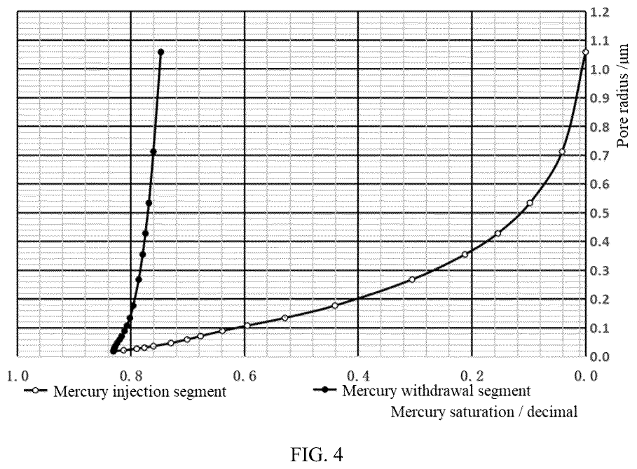

A first relation curve of the accumulated volume of mercury and the pore radius is plotted in a coordinate plot (as shown in FIG. 3), and according to the pore radius distribution intervals, the accumulated volume of injected mercury and the accumulated volume of withdrawn mercury at two end points of each pore radius distribution interval are determined in the first relation curve; and/or a second relation curve of the mercury saturation and the pore radius is plotted in a coordinate plot (as shown in FIG. 4), and according to the pore radius distribution intervals, the saturation of injected mercury and the saturation of withdrawn mercury at two end points of each pore radius distribution interval are determined in the second relation curve. The obtained data is shown in table 4.

TABLE 4

| | | Accumulated | Accumulated | | |
|---|---|---|---|---|---|
| | | volume of | volume of | | |
| | One end | mercury at | mercury at | Mercury | Mercury |
| | point of | end point of | end point of | saturation at | saturation at |
| | pore radius | mercury | mercury | end point of | end point of |
| Microscopic | distribution | injection | withdrawal | mercury | mercury |
| pore | interval | segment | segment | injection | withdrawal |
| number i | (μm) | (cm³) | (cm³) | segment | segment |
| 1 | 1.6 | 0.00000 | 1.68787 | 0.00000 | 0.74717 |
| 2 | 1 | 0.01581 | 1.69292 | 0.00700 | 0.74941 |
| 3 | 0.8 | 0.06977 | 1.71012 | 0.03089 | 0.75702 |
| 4 | 0.63 | 0.15238 | 1.72589 | 0.06746 | 0.76400 |
| 5 | 0.4 | 0.39861 | 1.75348 | 0.17645 | 0.77622 |
| 6 | 0.25 | 0.74822 | 1.78007 | 0.33122 | 0.78799 |
| 7 | 0.16 | 1.07356 | 1.80291 | 0.47524 | 0.79810 |
| 8 | 0.1 | 1.38212 | 1.82641 | 0.61183 | 0.80850 |
| 9 | 0.08 | 1.48509 | 1.83784 | 0.65741 | 0.81356 |
| 10 | 0.063 | 1.56523 | 1.84897 | 0.69288 | 0.81849 |
| 11 | 0.04 | 1.68924 | 1.86613 | 0.74778 | 0.82609 |
| 12 | 0.025 | 1.80052 | 1.87435 | 0.79704 | 0.82972 |
| 13 | 0.01779 | 1.87662 | 1.87662 | 0.83073 | 0.83073 |

Based on the data in tables 1 and 4, rock microscopic physical parameters are calculated.

Specifically, based on the data in table 4, the microscopic pore volume, the microscopic pore frequency, and the microscopic porosity for each microscopic pore in 5 the rock are calculated by equations (1) to (13). The obtained data is shown in table 5.

TABLE 5

| | | Microscopic | | | | | |
|---|---|---|---|---|---|---|---|
| | Pore throat | pore | | | | | |
| | radius | volume | Microscopic pore | | | | |
| Microscopic | distribution | Equations | frequency | | Microscopic porosity | | |
| pore | interval | (1) to (2) | Equations | Equations | Equations | Equations | Equation |
| number i | (μm) | (cm³) | (5) to (6) | (7) to (8) | (9) to (10) | (11) to (12) | (13) |
| 1 | 1.6-1.0 | 0.016 | 0.00708 | 0.007 | 0.00131 | 0.0013 | 0.0013 |
| 2 | 1.0-0.8 | 0.054 | 0.0239 | 0.02389 | 0.00442 | 0.00442 | 0.00442 |
| 3 | 0.8-0.63 | 0.083 | 0.03674 | 0.03657 | 0.0068 | 0.00677 | 0.00677 |
| 4 | 0.63-0.40 | 0.246 | 0.1089 | 0.109 | 0.02015 | 0.02017 | 0.02017 |
| 5 | 0.4-0.25 | 0.35 | 0.15494 | 0.15476 | 0.02866 | 0.02863 | 0.02863 |
| 6 | 0.25-0.16 | 0.325 | 0.14387 | 0.14402 | 0.02662 | 0.02664 | 0.02664 |
| 7 | 0.16-0.10 | 0.309 | 0.13679 | 0.13659 | 0.02531 | 0.02527 | 0.02527 |
| 8 | 0.10-0.08 | 0.103 | 0.0456 | 0.04558 | 0.00844 | 0.00843 | 0.00843 |
| 9 | 0.08-0.063 | 0.08 | 0.03541 | 0.03548 | 0.00655 | 0.00656 | 0.00656 |
| 10 | 0.063-0.04 | 0.124 | 0.05489 | 0.0549 | 0.01015 | 0.01016 | 0.01016 |
| 11 | 0.04-0.025 | 0.111 | 0.04914 | 0.04926 | 0.00909 | 0.00911 | 0.00911 |
| 12 | 0.025-0.016 | 0.076 | 0.03364 | 0.03369 | 0.00622 | 0.00623 | 0.00623 |
| 13 | 0-0.016 | 0.382 | 0.1691 | 0.16926 | 0.03128 | 0.03131 | 0.03131 |

| | | | | | | |
|---|---|---|---|---|---|---|
| Total | 2.259 | 1 | 1 | 0.185 | 0.185 | 0.185 |

Further, based on the data in table 5 the tortuosity for each microscopic pore in the rock is calculated by equations (14) to (15). The obtained data is shown in table 6.

TABLE 6

| | Pore throat | | | |
|---|---|---|---|---|
| | radius | | Microscopic tortuosity | |
| Microscopic | distribution | | (dimensionless) | |
| pore | interval | Microscopic | Equation | Equation |
| number i | (μm) | porosity | (14) | (15) |
| 1 | 1.6-1.0 | 0.00131 | 1.721 | 1.736 |
| 2 | 1.0-0.8 | 0.00442 | 1.695 | 1.702 |
| 3 | 0.8-0.63 | 0.0068 | 1.674 | 1.676 |
| 4 | 0.63-0.4 | 0.02015 | 1.555 | 1.532 |
| 5 | 0.4-0.25 | 0.02866 | 1.474 | 1.44 |
| 6 | 0.25-0.16 | 0.02662 | 1.494 | 1.462 |
| 7 | 0.16-0.10 | 0.02531 | 1.506 | 1.476 |
| 8 | 0.10-0.08 | 0.00844 | 1.660 | 1.659 |
| 9 | 0.08-0.063 | 0.00655 | 1.677 | 1.679 |
| 10 | 0.063-0.04 | 0.01015 | 1.645 | 1.64 |
| 11 | 0.04-0.025 | 0.00909 | 1.655 | 1.652 |
| 12 | 0.025-0.016 | 0.00622 | 1.679 | 1.683 |
| 13 | 0-0.016 | 0.03128 | / | / |

Further, based on the data in table 6, the permeability for each microscopic pore in the rock is calculated by equations (16) to (18). The obtained data is shown in table 7.

TABLE 7

| | | | | One end | The other | | | |
|---|---|---|---|---|---|---|---|---|
| | | | | point value | end point | | | |
| | Pore throat | | | of pore | value of pore | | | |
| | radius | | | radius | radius | Microscopic permeability | | |
| Microscopic | distribution | | Microscopic | distribution | distribution | (10⁻³ μm²) | | |
| pore | interval | Microscopic | tortuosity | interval | interval | Equation | Equation | Equation |
| number i | (μm) | porosity | (dimensionless) | (μm) | (μm) | (16) | (17) | (18) |
| 1 | 1.6-1 | 0.00131 | 1.721 | 1.05861 | 1.0 | 0.257 | 0.238 | 0.278 |
| 2 | 1-0.8 | 0.00442 | 1.695 | 1.0 | 0.8 | 0.674 | 0.632 | 0.716 |
| 3 | 0.8-0.63 | 0.0068 | 1.674 | 0.8 | 0.63 | 0.662 | 0.629 | 0.695 |
| 4 | 0.63-0.4 | 0.02015 | 1.555 | 0.63 | 0.4 | 1.096 | 1.12 | 1.069 |
| 5 | 0.4-0.25 | 0.02866 | 1.474 | 0.4 | 0.25 | 0.655 | 0.706 | 0.605 |
| 6 | 0.25-0.16 | 0.02662 | 1.494 | 0.25 | 0.16 | 0.239 | 0.254 | 0.224 |
| 7 | 0.16-0.1 | 0.02531 | 1.506 | 0.16 | 0.10 | 0.091 | 0.096 | 0.086 |
| 8 | 0.1-0.08 | 0.00844 | 1.66 | 0.10 | 0.08 | 0.013 | 0.013 | 0.014 |
| 9 | 0.08-0.063 | 0.00655 | 1.677 | 0.08 | 0.063 | 0.006 | 0.006 | 0.007 |
| 10 | 0.063-0.04 | 0.01015 | 1.645 | 0.063 | 0.04 | 0.005 | 0.005 | 0.005 |
| 11 | 0.04-0.025 | 0.00909 | 1.655 | 0.04 | 0.025 | 0.002 | 0.002 | 0.002 |
| 12 | 0.025-0.016 | 0.00622 | 1.679 | 0.025 | 0.01779 | 0.001 | 0.001 | 0.001 |

| | | |
|---|---|---|
| Total | 0.1537 | 3.7 |

Further, based on the data in tables 1 and 5, the fluid saturation for each microscopic pore in the rock is calculated by equations (19) to (21). The obtained data is shown in table 8.

TABLE 8

| | Pore throat | | | | |
|---|---|---|---|---|---|
| | radius | | | | |
| Microscopic | distribution | Microscopic | Microcosmic | Microcosmic | Microcosmic |
| pore | interval | porosity | gas | oil | water |
| number i | (μm) | (%) | saturation | saturation | saturation |
| 1 | 1.6-1.0 | 0.00131 | 1 | 0 | 0 |
| 2 | 1.0-0.8 | 0.00442 | 1 | 0 | 0 |
| 3 | 0.8-0.63 | 0.0068 | 1 | 0 | 0 |
| 4 | 0.63-0.4 | 0.02015 | 1 | 0 | 0 |
| 5 | 0.4-0.25 | 0.02866 | 0.409 | 0.591 | 0 |
| 6 | 0.25-0.16 | 0.02662 | 0 | 1 | 0 |
| 7 | 0.16-0.10 | 0.02531 | 0 | 1 | 0 |
| 8 | 0.10-0.08 | 0.00844 | 0 | 1 | 0 |
| 9 | 0.08-0.063 | 0.00655 | 0 | 1 | 0 |
| 10 | 0.063-0.04 | 0.01015 | 0 | 0.123 | 0.877 |
| 11 | 0.04-0.025 | 0.00909 | 0 | 0 | 1 |
| 12 | 0.025-0.016 | 0.00622 | 0 | 0 | 1 |
| 13 | 0-0.016 | 0.03128 | 0 | 0 | 1 |

Further, based on the data in tables 4 and 5, the efficiency of mercury withdrawal for each microscopic pore in the rock is calculated by equations (25) to (26). The obtained data is shown in table 9.

TABLE 9

| | Pore radius | | Efficiency of mercury | |
|---|---|---|---|---|
| Microscopic | distribution | | withdrawal (decimal) | |
| pore | interval | Microscopic | Equation | Equation |
| number i | (um) | porosity | (25) | (26) |
| 1 | 1.6-1 | 0.00131 | 0.3125 | 0.31857 |
| 2 | 1-0.8 | 0.00442 | 0.31481 | 0.31896 |
| 3 | 0.8-0.63 | 0.0068 | 0.19277 | 0.19087 |
| 4 | 0.63-0.4 | 0.02015 | 0.11382 | 0.11211 |
| 5 | 0.4-0.25 | 0.02866 | 0.07714 | 0.07605 |
| 6 | 0.25-0.16 | 0.02662 | 0.07077 | 0.0702 |
| 7 | 0.16-0.1 | 0.02531 | 0.07443 | 0.07614 |
| 8 | 0.1-0.08 | 0.00844 | 0.1068 | 0.11101 |
| 9 | 0.08-0.063 | 0.00655 | 0.1375 | 0.13895 |
| 10 | 0.063-0.04 | 0.01015 | 0.1371 | 0.13843 |
| 11 | 0.04-0.025 | 0.00909 | 0.07207 | 0.07389 |
| 12 | 0.025-0.016 | 0.00622 | 0.02632 | 0.02998 |

In the above description of this application, the terms “fixed”, “installed”, “connected” or “coupled” or the like are to be construed broadly, unless otherwise expressly stated and defined. For example, the term “connected” may refer to components being fixedly connected, detachably connected, or forming an integral; or being mechanically connected or electrically connected; or being directly connected, indirectly connected via an intermedium, or two elements in internal communication or interaction. Therefore, those skilled in the art may understand the specific meanings of the above terms in the present disclosure according to the specific context, unless otherwise explicitly limited in the present application.

In addition, “first”, “second” or other terms for indicating a number or sequence used herein are merely for the purpose of illustration and are not to be construed as indicating or implying a relative importance or implicitly indicating the number of the indicated technical features. Therefore, a feature defined by “first” or “second” may include at least one that feature either explicitly or implicitly. In the description of the present application, “a plurality of” means at least two, e.g., two, three or more, unless explicitly defined otherwise.

Although various embodiments of the present disclosure have been shown and described herein, it will be obvious to those skilled in the art that such embodiments are provided by way of example only. Numerous modifications, changes, and substitutions may occur to those skilled in the art without departing from the spirit and scope of the present disclosure. It should be appreciated that various alternatives to the embodiments of the present disclosure described herein may be employed in practicing the present disclosure. It is intended that the following claims define the scope of the present disclosure and that equivalents or alternatives within the scope of these claims are covered thereby.

What is claimed is:

1. A method for determining rock microscopic physical parameters, the method comprising:
acquiring macroscopic physical parameters of a rock, wherein the macroscopic physical parameters includes an apparent volume, a pore volume, a porosity, a permeability and a fluid saturation of the rock;
acquiring capillary pressure curve experimental data of the rock, wherein the capillary pressure curve experimental data includes a capillary pressure, an accumulated volume of a nonwetting phase fluid, and/or a saturation of a nonwetting phase fluid;
based on the capillary pressure curve experimental data of the rock, determining an accumulated volume of a nonwetting phase fluid at two end points of different pore radius distribution intervals of the rock, and/or determining a saturation of the nonwetting phase fluid at the two end points of the different pore radius distribution intervals of the rock; and
calculating the rock microscopic physical parameters based on the macroscopic physical parameters of the rock, the accumulated volume of the nonwetting phase fluid at the two end points of the different pore radius distribution intervals of the rock, and/or the saturation of the nonwetting phase fluid at the two end points of the different pore radius distribution intervals of the rock, wherein the microscopic physical parameters includes a microscopic pore volume, a microscopic porosity, a microscopic tortuosity, a microscopic permeability, a microscopic fluid saturation, and/or a microscopic efficiency of mercury withdrawal of the rock, wherein microscopic pores include microscopic pores with saturated nonwetting phase fluid and microscopic pores with minimum unsaturated nonwetting phase fluid, and wherein the calculating of the rock microscopic physical parameters includes dividing the rock into a plurality of microscopic pores.

2. The method of claim 1, wherein the microscopic pore volume is calculated by one of:
a first method in which:
for an i^{th} microscopic pore with saturated nonwetting phase fluid in the rock, the pore volume is calculated by:
$${V}_{P i} = {V}_{H g j ( i + 1 )} - {V}_{H g j i} = Δ {V}_{H g j i} \qquad ( 1 )$$
where: V_{Pi} is the volume of the i^{th} microscopic pore in the rock; V_{Hgji} is the accumulated volume of the nonwetting phase fluid in the rock corresponding to one end point value rᵢ of an i^{th} pore radius distribution interval; V_{Hgj(i+1)} is the accumulated volume of the nonwetting phase fluid in the rock corresponding to the other end point value rᵢ₊₁ of the i^{th} pore radius distribution interval; and ΔV_{Hgji} is the volume of the nonwetting phase fluid injected into the i^{th} microscopic pore in the rock, where i is a sequence number, and i∈[1,n−1]; n is the total number of microscopic pores divided from the rock according to the pore radius distribution intervals; r; and rᵢ₊₁ are pore radii, and rᵢ₊₁<rᵢ; and
for a microscopic pore with minimum unsaturated nonwetting phase fluid in the rock which is an n^{th} microscopic pore, the pore volume is calculated by:
$${V}_{P n} = {V}_{P} - {V}_{H g j n} = {V}_{P} - {\sum }_{1}^{n - 1} {V}_{p i} \qquad ( 2 )$$
where: V_{Pn} is the volume of the n^{th} microscopic pore in the rock; V_{P} is the pore volume of the rock; and V_{Hgjn} is the accumulated volume of the nonwetting phase fluid injected into the rock at a maximum injection pressure; or
a second method in which:
for an i^{th} microscopic pore with saturated nonwetting phase fluid in the rock, the pore volume is calculated by:
$${V}_{P i} = ( {S}_{H g j ( i + 1 )} - {S}_{H g j i} ) {V}_{P} = Δ {S}_{H g j i} {V}_{P} \qquad ( 3 )$$
where: V_{Pi} is the volume of the i^{th} microscopic pore in the rock; S_{Hgji} is the saturation of the nonwetting phase fluid injected into the rock corresponding to one end point value rᵢ of the i^{th} pore radius distribution interval; S_{Hgj(i+1)} is the saturation of the nonwetting phase fluid injected into the rock corresponding to the other end point value rᵢ₊₁ of the i^{th} pore radius distribution interval; ΔS_{Hgji} is the saturation of the nonwetting phase fluid injected into the i^{th} microscopic pore in the rock; and V_{P} is the pore volume of the rock, where i is a sequence number, and i∈[1, n−1]; n is the total number of microscopic pores divided from the rock according to the pore radius distribution intervals; r; and rᵢ₊₁ are pore radii, and rᵢ₊₁<rᵢ; and
for a microscopic pore with minimum unsaturated nonwetting phase fluid in the rock, which is an n^{th} microscopic pore, the pore volume is calculated by:
$${V}_{P n} = ( 1 - {S}_{H g j n} ) {V}_{P} \qquad ( 4 )$$
where: V_{Pn} is the volume of the n^{th} microscopic pore in the rock; V_{P} is the pore volume of the rock; and S_{Hgjn} is the saturation of the nonwetting phase fluid injected into the rock at a maximum injection pressure.

3. The method of claim 1, wherein the microscopic pore frequency is calculated by one of:
a first method in which:
for an i^{th} microscopic pore with saturated nonwetting phase fluid in the rock, the pore frequency is calculated by:
$${f}_{i} = \frac{{V}_{Hgj ( i + 1 )} - {V}_{Hgji}}{{V}_{P}} = \frac{Δ {V}_{Hgji}}{{V}_{P}} \qquad ( 5 )$$
where: fᵢ is the frequency of the i^{th} microscopic pore in the rock; V_{Hgji} is the accumulated volume of the nonwetting phase fluid in the rock corresponding to one end point value rᵢ of an i^{th} pore radius distribution interval; V_{Hgj(i+1)} is the accumulated volume of the nonwetting phase fluid in the rock corresponding to the other end point value rᵢ₊₁ of the i^{th} pore radius distribution interval; ΔV_{Hgji} is the volume of the nonwetting phase fluid injected into the i^{th} microscopic pore in the rock; V_{P} is the pore volume of the rock; and n is the total number of microscopic pores divided from the rock according to the pore radius distribution intervals, where i is a sequence number, and i∈[1, n−1]; and rᵢ and rᵢ₊₁ are pore radii, and rᵢ₊₁<rᵢ; and
for a microscopic pore with minimum unsaturated nonwetting phase fluid in the rock, which is an n^{th} microscopic pore, the pore frequency is calculated by:
$${f}_{n} = \frac{{V}_{P} - {V}_{Hgjn}}{{V}_{P}} \qquad ( 6 )$$
where: fₙ is the frequency of the n^{th} microscopic pore in the rock; V_{P} is the pore volume of the rock; and V_{Hgjn} is the accumulated volume of the nonwetting phase fluid injected into the rock at a maximum injection pressure; or
a second method in which:
for an i^{th} microscopic pore with saturated nonwetting phase fluid in the rock, the pore frequency is calculated by:
$${f}_{i} = {S}_{Hgj ( i + 1 )} - {S}_{Hgji} = Δ {S}_{Hgji} \qquad ( 7 )$$
where: fᵢ is the frequency of the i^{th} microscopic pore in the rock; S_{Hgji} is the saturation of the nonwetting phase fluid injected into the rock corresponding to one end point value rᵢ of the i^{th} pore radius distribution interval; S_{Hgj(i+1)} is the saturation of the nonwetting phase fluid injected into the rock corresponding to the other end point value rᵢ₊₁ of the i^{th} pore radius distribution interval; ΔS_{Hgji} is the saturation of the nonwetting phase fluid injected into the i^{th} microscopic pore in the rock; and n is the total number of microscopic pores divided from the rock according to the pore radius distribution intervals; where i is a sequence number, and i∈[1, n−1]; and rᵢ and rᵢ₊₁ are pore radii, and rᵢ₊₁<rᵢ; and
for a microscopic pore with minimum unsaturated nonwetting phase fluid in the rock, which is an n^{th} microscopic pore, the pore frequency is calculated by:
$${f}_{n} = 1 - {S}_{Hgjn} = 1 - {\sum }_{1}^{n - 1} {f}_{i} \qquad ( 8 )$$
where: fₙ is the frequency of the n^{th} microscopic pore in the rock; and S_{Hgjn} is the saturation of the nonwetting phase fluid injected into the rock at a maximum injection pressure.

4. The method of claim 1, wherein the microscopic porosity is calculated by one of:
a first method in which:
for an i^{th} microscopic pore with saturated nonwetting phase fluid in the rock, the porosity is calculated by:
$${∅}_{i} = \frac{{V}_{Hgj ( i + 1 )} - {V}_{Hgji}}{{V}_{b}} = \frac{Δ {V}_{Hgji}}{{V}_{b}} \qquad ( 9 )$$
where: Øᵢ is the porosity of the i^{th} microscopic pore in the rock; V_{P} is the apparent volume of the rock; V_{Hgji} is the accumulated volume of the nonwetting phase fluid in the rock corresponding to one end point value rᵢ of an i^{th} pore radius distribution interval; V_{Hgj(i+1)} is the accumulated volume of the nonwetting phase fluid in the rock corresponding to the other end point value rᵢ₊₁ of the i^{th} pore radius distribution interval; ΔV_{Hgji} is the volume of the nonwetting phase fluid injected into the i^{th} microscopic pore in the rock; and n is the total number of microscopic pores divided from the rock according to the pore radius distribution intervals; where i is a sequence number, and i∈[1, n−1]; and rᵢ and rᵢ₊₁ are pore radii, and rᵢ₊₁<rᵢ; and
for a microscopic pore with minimum unsaturated nonwetting phase fluid in the rock, which is an n^{th} microscopic pore, the porosity is calculated by:
$${∅}_{n} = \frac{{V}_{b} - {V}_{Hgjn}}{{V}_{b}} \qquad ( 10 )$$
where: Øₙ is the porosity of the n^{th} microscopic pore in the rock; V_{P} is the apparent volume of the rock; V_{Hgjn} is the accumulated volume of the nonwetting phase fluid injected into the rock at a maximum injection pressure;
a second method in which:
for an i^{th} microscopic pore with saturated nonwetting phase fluid in the rock, the porosity is calculated by:
$${∅}_{i} = ( {S}_{Hgj ( i + 1 )} - {S}_{Hgji} ) ∅ = Δ {S}_{Hgji} ∅ \qquad ( 11 )$$
where: Øᵢ is the porosity of the i^{th} microscopic pore in the rock; Ø is the porosity of the rock; S_{Hgji} is the saturation of the nonwetting phase fluid injected into the rock corresponding to one end point value rᵢ of the i^{th} pore radius distribution interval; S_{Hgj(i+1)} is the saturation of the nonwetting phase fluid injected into the rock corresponding to the other end point value rᵢ₊₁ of the i^{th} pore radius distribution interval; ΔS_{Hgji} is the saturation of the nonwetting phase fluid injected into the i^{th} microscopic pore in the rock; and n is the total number of microscopic pores divided from the rock according to the pore radius distribution intervals; where i is a sequence number, and i∈[1, n−1]; and rᵢ and rᵢ₊₁ are pore radii, and rᵢ₊₁<rᵢ; and
for a microscopic pore with minimum unsaturated nonwetting phase fluid in the rock, which is an n^{th} microscopic pore, the porosity is calculated by:
$${∅}_{n} = ( 1 - {S}_{Hgjn} ) ∅ = ∅ - {\sum }_{1}^{n - 1} {∅}_{i} \qquad ( 12 )$$
where: Øₙ is the porosity of the n^{th} microscopic pore in the rock; Ø is the porosity of the rock; Øᵢ is the porosity of the i^{th} microscopic pore in the rock; and
S_{Hgjn} is the saturation of the nonwetting phase fluid injected into the rock at a maximum injection pressure, where i is a sequence number, and i∈[1, n−1]; or
a third method in which:
the porosity of an i^{th} microscopic pore in the rock is calculated by:
$${∅}_{i} = {f}_{i} ∅ \qquad ( 13 )$$
where: Øᵢ is the porosity of the i^{th} microscopic pore in the rock; fᵢ is the frequency of the i^{th} microscopic pore in the rock; and Ø is the porosity of the rock; where i is a sequence number, and i∈[1, n]; and n is the total number of microscopic pores divided from the rock according to the pore radius distribution intervals.

5. The method of claim 1, wherein the microscopic tortuosity is calculated by one of:
a first method in which for an i^{th} microscopic pore in the rock, the tortuosity is calculated by:
$${τ}_{i} = \sqrt{1 + 2 ( 1 - \frac{( n - 1 ) {∅}_{i}}{{\sum }_{1}^{n - 1} {∅}_{i}} ∅ )} \qquad ( 14 )$$
where: τᵢ is the tortuosity of the i^{th} microscopic pore in the rock; Ø is the porosity of the rock; and Øᵢ is the porosity of the i^{th} microscopic pore in the rock;
where i is a sequence number, and i∈[1, n−1]; and n is the total number of microscopic pores divided by the rock according to the pore radius distribution intervals;
a second method in which for an i^{th} microscopic pore in the rock, the tortuosity is calculated by:
$${τ}_{i} = 1 + α ( 1 - \frac{( n - 1 ) {∅}_{i}}{{\sum }_{1}^{n - 1} {∅}_{i}} ∅ ) \qquad ( 15 )$$
where: τᵢ is the tortuosity of the i^{th} microscopic pore in the rock; a is an equation coefficient; Ø is the porosity of the rock; and Øᵢ is the porosity of the i^{th} microscopic pore in the rock; where i is a sequence number, and i∈[1, n−1]; and
n is the total number of microscopic pores divided from the rock according to the pore radius distribution intervals.

6. The method of claim 1, wherein the microscopic permeability is calculated by:
for an i^{th} microscopic pore in the rock, the permeability is calculated by any one of the following equations (16) to (18):
$${k}_{i} = \frac{{( {r}_{i} + {r}_{i + 1} )}^{2} \frac{{∅}_{i}}{{τ}_{i}^{2}}}{{\sum }_{1}^{n - 1} {( {r}_{i} + {r}_{i + 1} )}^{2} \frac{{∅}_{i}}{{τ}_{i}^{2}}} K \qquad ( 16 )$$
$${k}_{i} = \frac{{( {r}_{i} + {r}_{i + 1} )}^{2} \frac{{∅}_{i}}{{τ}_{i}}}{{\sum }_{1}^{n - 1} {( {r}_{i} + {r}_{i + 1} )}^{2} \frac{{∅}_{i}}{{τ}_{i}}} K \qquad ( 17 )$$
$${k}_{i} = \frac{{( {r}_{i} + {r}_{i + 1} )}^{2} {∅}_{i}}{{\sum }_{1}^{n - 1} {( {r}_{i} + {r}_{i + 1} )}^{2} {∅}_{i}} K \qquad ( 18 )$$
where: kᵢ is the permeability the i^{th} microscopic pore in the rock; Øᵢ is the porosity of the i^{th} microscopic pore in the rock; rᵢ is the pore radius at one end point of an i^{th} pore radius distribution interval; rᵢ₊₁ is the pore radius at the other end point of the i^{th} pore radius distribution interval; τᵢ is the tortuosity of the i^{th} microscopic pore in the rock; and K is the permeability of the rock, where i is a sequence number, and i∈[1, n−1]; and n is the total number of microscopic pores divided from the rock according to the pore radius distribution intervals.

7. The method of claim 1, wherein the microscopic fluid saturation is calculated by:
acquiring measurement results of the porosity, the fluid saturation, the microscopic porosity or the microscopic pore frequency of the rock;
sequencing the microscopic pores in the rock according to sizes of pore radii;
obtaining filling sequences and filling capacities of different fluids in different pore throats;
preliminarily determining a value of the microscopic fluid saturation based on the filling sequences and the filling capacities of different fluids in different pore throats;
substituting a preliminary discrimination result of each microscopic fluid saturation in the rock into relational expressions (19), (20) and (21) of the fluid saturation, the porosity, the microscopic fluid saturation and the microscopic porosity; or into relational expressions (22), (23) and (24) of the fluid saturation, the microscopic fluid saturation and the microscopic pore frequency, to obtain distribution of fluid saturations in the microscopic pores by simultaneous solution and continuous trial calculation;
$${\sum }_{1}^{n} {∅}_{i} {S}_{gi} = ∅ {S}_{g} \qquad ( 19 )$$
$${\sum }_{1}^{n} {∅}_{i} {S}_{oi} = ∅ {S}_{o} \qquad ( 20 )$$
$${\sum }_{1}^{n} {∅}_{i} {S}_{wi} = ∅ {S}_{w} \qquad ( 21 )$$
$${\sum }_{1}^{n} {f}_{i} {S}_{gi} = {S}_{g} \qquad ( 22 )$$
$${\sum }_{1}^{n} {f}_{i} {S}_{oi} = {S}_{o} \qquad ( 23 )$$
$${\sum }_{1}^{n} {f}_{i} {S}_{wi} = {S}_{w} \qquad ( 24 )$$
where: Sₒᵢ is the oil saturation of the i^{th} microscopic pore in the rock; Sₒ is the oil saturation of the rock; S_{wi} is the water saturation of the i^{th} microscopic pore in the rock; S_{w} is the water saturation of the rock; S_{gi} is the gas saturation of the i^{th} microscopic pore in the rock; Sg is the gas saturation of the rock; fᵢ is the frequency of the i^{th} microscopic pore in the rock; Øᵢ is the porosity of the i^{th} microscopic pore in the rock; and Ø is the porosity of the rock; where i is a sequence number, and i∈[1, n]; and n is the total number of microscopic pores divided from the rock according to the pore radius distribution intervals.

8. The method of claim 1, wherein the microscopic efficiency of mercury withdrawal is calculated by one of:
a first method in which for a microscopic pore, the efficiency of mercury withdrawal is calculated by:
$${W}_{Ei} = \frac{{V}_{Hgt ( i + 1 )} - {V}_{Hgti}}{{V}_{Hgj ( i + 1 )} - {V}_{Hgji}} = \frac{Δ {V}_{Hgti}}{Δ {V}_{Hgji}} \qquad ( 25 )$$
where: W_{Ei} is the efficiency of mercury withdrawal of the i^{th} microscopic pore in the rock; V_{Hgji} is the accumulated volume of mercury in the rock corresponding to one end point rᵢ of the i^{th} pore radius distribution interval in a mercury feeding process; V_{Hgj(i+1)} is the accumulated volume of mercury in the rock corresponding to the other end point rᵢ₊₁ of the i^{th} pore radius distribution interval in the mercury feeding process; V_{Hgti} is the accumulated volume of mercury in the rock corresponding to one end point rᵢ of the i^{th} pore radius distribution interval in the rock in a mercury withdrawal process; V_{Hgt(i+1)} is the accumulated volume of mercury in the rock corresponding to the other end point rᵢ₊₁ of the i^{th} pore radius distribution interval in the rock in the mercury withdrawal process; ΔV_{Hgji} is the volume of mercury injected into the i^{th} microscopic pore in the rock in the mercury feeding process; and ΔV_{Hgti} is the volume of mercury withdrawn from the i^{th} microscopic pore in the rock in the mercury withdrawal process, where i is a sequence number, and i∈[1, n−1]; rᵢ and rᵢ₊₁ are pore radii, and rᵢ₊₁<rᵢ; and n is the total number of microscopic pores divided from the rock according to the pore radius distribution intervals; or
a second method in which for a microscopic pore, the efficiency of mercury withdrawal is calculated by:
$${W}_{Ei} = \frac{{S}_{Hgt ( i + 1 )} - {S}_{Hgti}}{{S}_{Hgj ( i + 1 )} - {S}_{Hgji}} = \frac{Δ {S}_{Hgti}}{Δ {S}_{Hgji}} \qquad ( 26 )$$
where: W_{Ei} is the efficiency of mercury withdrawal of the i^{th} microscopic pore in the rock; S_{Hgji} is the mercury saturation in the rock corresponding to one end point rᵢ of the i^{th} pore radius distribution interval in a mercury feeding process; S_{Hgj(i+1)} is the mercury saturation in the rock corresponding to the other end point rᵢ₊₁ of the i^{th} pore radius distribution interval in the mercury feeding process; S_{Hgti} is the mercury saturation in the rock corresponding to one end point rᵢ of the i^{th} pore radius distribution interval in a mercury withdrawal process; S_{Hgt(i+1)} is the mercury saturation in the rock corresponding to the other end point rᵢ₊₁ of the i^{th} pore radius distribution interval in the mercury withdrawal process; ΔS_{Hgji} is the saturation of mercury injected into the i^{th} microscopic pore in the rock in the mercury feeding process; and ΔS_{Hgti} is the saturation of mercury withdrawn from the i^{th} microscopic pore in the rock in the mercury withdrawal process; where i is a sequence number, and i∈[1, n−1]; rᵢ and rᵢ₊₁ are pore radii, and rᵢ₊₁<rᵢ; and n is the total number of microscopic pores divided from the rock according to the pore radius distribution intervals.

9. An apparatus for determining rock microscopic physical parameters, the apparatus comprising:
a first acquisition unit configured to acquire macroscopic physical parameters of a rock, wherein the macroscopic physical parameters includes an apparent volume, a pore volume, a porosity, a permeability and a fluid saturation of the rock;
a second acquisition unit configured to acquire capillary pressure curve experimental data of the rock, wherein the experimental data includes a capillary pressure, an accumulated volume of a nonwetting phase fluid, and/or a saturation of a nonwetting phase fluid;
a third acquisition unit configured to acquire an accumulated volume of a nonwetting phase fluid at two end points of different pore radius distribution intervals of the rock, and/or a saturation of the nonwetting phase fluid at the two end points of the different pore radius distribution intervals of the rock; and
a logical computation unit configured to calculate the rock microscopic physical parameters based on the macroscopic physical parameters of the rock, the accumulated volume of the nonwetting phase fluid at the two end points of the different pore radius distribution intervals of the rock, and/or the saturation of the nonwetting phase fluid at the two end points of the different pore radius distribution intervals of the rock, wherein the microscopic physical parameters includes a microscopic pore volume, a microscopic pore frequency, a microscopic porosity, a microscopic tortuosity, a microscopic permeability, a microscopic fluid saturation, and/or a microscopic efficiency of mercury withdrawal of the rock.

10. An electronic device for determining rock microscopic physical parameters, comprising a processor, and a memory storing executable program instructions thereon which, when executed by the processor, cause the electronic device to implement the method of claim 1.

11. A non-transitory computer-readable storage medium for determining rock microscopic physical parameters, wherein the computer-readable storage medium has a computer program stored thereon which, when executed by one or more processors, cause the one or more processors to implement the method of claim 1.
